(19) United States
(12) Patent Application Publication
(10) Pub. No.: US 2006/0200128 A1
(43) Pub. Date: Sep. 7, 2006

(54) Bone anchor

(76) Inventor: Richard Mueller, Macedonia, OH (US)

Correspondence Address: MIDDLETON & REUTLINGER, 2500 BROWN & WILLIAMSON TOWER, LOUISVILLE, KY, 40202, US

(21) Appl. No.: 10/551,101

(22) PCT Filed: Apr. 2, 2004
(86) PCT No.: PCT/US04/10319

§ 371 Date: Sep. 26, 2005

Related U.S. Application Data
(60) Provisional application No. 60/460,616, filed on Apr. 4, 2003.

(51) Int. Cl. A61F 2/30

(52) U.S. Cl. 606/61

(57) ABSTRACT

A spinal rod bone anchor (00) having a proximal end for engaging a rod (200) and securing the rod with a coming or threaded lock assembly (300) and a distal end for engaging bone. A locking device (300) received within two flanges (110, 120) having an upper wave cap (310) and a lower cap (330) joined by a post with preset timing grooves (335) therein and having camming surfaces therebetween (317, 338).

### BACKGROUND OF THE INVENTION

1. Field of the Invention

The present invention generally relates to the correction of spinal deformities. Specifically, the present invention provides an improved apparatus and method for maintaining vertebrae in a desired spatial relationship.

2. Background

The human spinal column is composed of many vertebral bones stacked one upon the other, with an intervertebral disc between each pair of adjacent vertebral bones. The discs act as cartilaginous cushions and shock absorbers. The spinal cord runs in a bony canal formed by successive openings in these bones. Spinal nerves exit the spinal cord between pairs of vertebrae and supply nerve signals to and from other body structures.

Various problems with the human spine have been encountered that adversely affect its health. These problems include spinal column disorders such as scoliosis, kyphosis, spondylolisthesis, as well as traumatic events such as ruptured or slipped discs, broken or fractured spinal columns, and the like. Various forms of instrumentation and procedures are known for the surgical treatment of spinal disorders, for example, Harrington Spinal Instrumentation, Bobechlco Hooks, Edwards Hooks and Rod Sleeves, Luque Segmental Spinal Instrumentation and Luque Rectangles, the Dunn Anterior Spinal System, and the Kostuik-Harrington Instrumentation.

The use of longitudinally extending surgical rods in the treatment of diseases or instability of the spine is well-known in the medical arts. Such rods achieve rigid spinal fixation when mechanically coupled to bone anchors, such as hooks or screws. These surgical rods are used, generally, in pairs placed on the posterior surface of the left and right sides of the lamina of the human spine.

Some of the above systems utilize hook-type members which are merely hooked over the laminae or on selected transverse processes of the spine. Other systems, such as the Luque Segmental Spinal Rectangles, used to stabilize spinal fractures and low back fusions, use Luque wires to secure the rectangle to the spine. In some of the prior art systems, screws are used to hold a single rod in place. In other systems, screws are used to hold a slotted plate in place, the location of the screws and slots being such that the plate is moved in order to align the plate apertures or slots with the end of the screw, a nut being used to hold the plate to the screw. With this latter arrangement there is little purchase between the plate and the screw and nut since only a small portion of the plate is engaged adjacent to the slots. Also, the plate cannot be configured to a fixed and stable curvature to follow the curvature desired by the surgeon.

Another known corrective device includes a plurality of plates. Each of the plates is secured over one end of a vertebra. Fasteners are connected to the vertebrae through the plates. A cable is then crimped in the head of the fastener to attach the cable to one vertebra. Tension is put on the cable while it is crimped to an adjacent vertebra until the desired correction is accomplished. This device can only put compressive forces on the spine so that the cables are always in tension. Once the cable is crimped in place, no further adjustment between the crimped fastener and cable is possible.

In devices utilizing rods, the corrective forces are generated by (usually) two rods that are wired around the spine. The rods may be bent to a desired curvature. The rods are not directly attached to all the vertebrae that the rods span; rather, they span numerous vertebrae and are connected to only a few vertebrae using anchors, generally hooks or screws.

One widely used anchor for rod systems is the conventional orthopedic hook having a block-shaped head portion, with a central, cylindrical bore therethrough, and a hook portion. The bore of the conventional orthopedic hook is adapted to receive the surgical rod, and the head is slidably positioned over the surface of the surgical rod to the selected vertebra for attachment. The hook may have a variety of different shapes, lengths and openings to accommodate the specific vertebra to which it is to be anchored. With the hook portion properly anchored, the conventional orthopedic hook is locked to the surgical rod either by ratchet or by one or more set screws located within the block-shaped head. However, these systems do not provide polyaxial alignments of the anchors. Rather, the anchors are fixed in a given orientation with respect to the bone and allow no movement in vivo or in response to applied loads.

Another type of anchor is a special orthopedic screw having a block-shaped head with cylindrical bore therethrough. The screw, when its threaded end is attached to the selected anatomical site, is adapted for receiving and passing the elongated surgical rod through its cylindrical bore. Since the shank and threaded end of the screw extends perpendicularly with respect to the axis of the bore, once the screw has been anchored, the position of the head, with its cylindrical bore, is fixed with respect to the Spine of the patient.

If the nature of the disease of the spine should require the attachment of a number of orthopedic screws at spaced-apart anatomical sites, it will be appreciated that manual insertion of an elongated surgical rod through the bores of the several spaced-apart orthopedic screws is surgically difficult. The alignment of the axis of the bore in the head of each screw must, of necessity, bear some relationship to a common axis related to the axis of the surgical rod, which rod must be inserted through the several bores. Since the nature of the surgical operation places the surgical rod under stress, as by resisting deforming forces of the spine, it will be appreciated that proper positioning of the heads and alignment of the bores of the several anchor attachment members is of paramount concern.

Some systems have attempted to provide bone screw/rod anchor devices that include polyaxial screws, with varying degrees of success. Most systems that attempt to provide for polyaxial capabilities employ a spherical head or ball-shaped head for the screw. While this allows angulation, it also provides an undesirable structure as the spherical head takes up too much space in the construct. Moreover, such systems rely on a locking screw to apply a compressive force between the lower surface of the rod and the upper surface of the ball to “lock” the angle of entry of the screw. Several disadvantages have been noted with these systems. The threaded locking screw is cumbersome and can become cross threaded, thus ruining the anchor that has already been inserted into the bone. The caps have internal threads that engage external threads on the anchor seat. This arrangement is cumbersome and inefficient from space-savings standpoint. Threaded connections provide no indication when tightening is completed; thus, overthreading is likely, which results in a connection that is not secure. By using a set screw without the “saddle” type of contact on the rod created by the present invention, two separate detrimental effects occur. First, just having single point contact, as a flat bottom set-screw meets at only one point with a circular rod, provides little to no holding force. The “saddle” contact area of the present invention, which is nearly half the circumference of the rod, provides a great deal of holding force. Second, as a result of the single point contact, rod “notching” can occur because all the axial force of driving the set screw is transferred into a single point on the rod. This notch created in the rod to hold the two together creates an instant stress riser, which is a failure point for systems using that type of design.

Recognizing that the surgical rods, with their anchor attachments, are installed during open-back surgery, while the patient is under anesthesia, it is important that the orthopedic surgeon have available for immediate use anchor attachments that are easily positioned and aligned between the vertebrae and the surgical rods to which they are to be secured. A device is needed that accommodates both angularly adjustable anchor attachment members as well as non-angularly adjustable anchor attachment members for mechanical intercoupling between selected vertebrae of the spine and a surgical rod. In addition, a device is needed that provides the surgeon with a simple, effective, lock that optionally can provide the surgeon with feedback as to the status of the lock situation. All of these features are needed in a low profile device that is space-efficient.

### BRIEF SUMMARY OF THE INVENTION

The present invention is directed to an anchor assembly for the internal fixation of the spine. The anchor assembly in several embodiments provides a quick-connect head and quick-disconnect cam locking cap to allow surgeons the ability to quickly and simply lock the anchor assembly to spinal rods or other stabilization means. In other embodiments the anchor assembly provides the surgeon with a simple threaded head that can be timed with the saddle to prevent cross-threading of the

In a first embodiment of the anchor assembly, the apparatus comprises a non-polyaxial bone anchor having a distal end for engaging a bone and a proximal end for engaging a surgical rod. The proximal end further comprises first and second opposed flanges defining a channel therebetween. The channel has a circular bottom surface for receiving a surgical rod. A radially-extending undercut having a leading surface and a trailing surface is disposed in the first and second opposed flanges for receiving a camming surface of a quick-connect cam device. The trailing surface further comprises a protruding portion to serve as a stop for engaging timing stops of the cam device.

The cam device comprises an upper portion and a lower portion. The upper portion comprises a substantially cylindrical body with an upper surface and a lower surface and an outer surface and a drive opening in said upper surface for receiving a drive mechanism (such as a hex drive, a Torx drive, and so forth). The drive opening has a bottom surface. A longitudinal opening extends through the bottom surface of the drive opening to receive a post wave cap. The outer surface of the upper portion further comprises radially extending flanges oriented approximately 180 degrees from each other for engaging the undercut within the first and second opposed flanges. Preferably the outer surface also comprises two timing stops, also oriented 180 degrees from each other but preferably offset from the radially extending flanges. The timing stops allow the upper portion to be turned 90 degrees (but no farther) inside the undercut. The anchor is inserted into the bone using an engaging device. A rod is placed in the channel in the appropriate orientation and a cam device is placed in the channel atop the rod. A drive instrument is inserted into the cam and a torque is applied to turn the cam 90 degrees to lock it into place.

In a second embodiment of the anchor assembly, the apparatus comprises a polyaxial anchor having a distal end for engaging a bone and a proximal end for engaging a rod. The proximal end comprises a head having a lower curvate surface and an upper low profile nonspherical surface. A top cap is provided having an upper portion for receiving the cam device and a lower portion for receiving the head of the polyaxial anchor. In addition, a bottom ring is provided that surrounds the lower portion of the top cap. The lower portion has fingers thereon that provide a radial force to help lock the angle of the anchor when the bottom ring is moved from its unlocked position to its locked position.

An alternative embodiment of the cam device is provided wherein the flanges have an upwardly extending ridge thereon that mates with an upwardly extending opening in the undercut. Once the ridges engage the upwardly extending opening, splaying of the flanges is prevented.

Another embodiment provides a two-piece threaded cap assembly in place of the cam cap assembly. The thread is preferably of buttress design and can have single or double start threads. The lower cap is similar to the lower cap of the cam embodiments. The threads can be timed with the lower surface of the lower cap so that when the lower surface of the lower cap is in position to receive a rod, the start threads are line up for automatic threading to prevent cross threading.

### BRIEF DESCRIPTION OF THE DRAWINGS

The teachings of the present invention can be readily understood by considering the following detailed description in conjunction with the accompanying drawings, in which:

FIG. 1 is a perspective view of the apparatus according to a first non-polyaxial embodiment;

FIG. 2 is a perspective view of the apparatus of FIG. 1 having removed the rod and the cam assembly, showing a first non-polyaxial bone anchor embodiment;

FIG. 3 is a top view of the bone anchor of FIG. 2;

FIG. 4 is a side view in a first radial direction of the bone anchor of FIG. 2;

FIG. 5 is a side view in a second radial direction of the bone anchor of FIG. 2, wherein the second radial direction is rotated 90 degrees from the first radial direction;

FIG. 6 is a perspective view of the partially cylindrical body of a first non-polyaxial embodiment of the invention;

FIG. 7 is a section view of the partially cylindrical body of FIG. 6 in a first radial direction;

FIG. 8 is a section view of the partially cylindrical body shown in FIG. 7 in a second radial direction;

FIG. 9 is a perspective view of the cam assembly showing upper wave cap and lower cap in mating contact;

FIG. 10 is a side elevation view of the cam assembly of FIG. 9 taken from a first radial direction;

FIG. 11 is a side elevation view of the cam assembly of FIG. 9 taken from a second radial direction;

FIG. 12 is a top view of the cam assembly;

FIG. 13 is a bottom view of the cam assembly;

FIG. 14 is a perspective view of the cam assembly of FIG. 9, shown with the upper wave cap having been rotated 90 degrees;

FIG. 15 is a perspective view of the upper wave cap;

FIG. 16 is a perspective view of the lower cap;

FIG. 17 is a top view of the apparatus showing the cam assembly in the unlocked position;

FIG. 18 is a top view of the apparatus showing the cam assembly in the locked position;

FIG. 19 is a perspective view of the apparatus according to a first polyaxial embodiment;

FIG. 20 is a top view of an embodiment of a polyaxial bone anchor, shown as a screw, for use in the apparatus;

FIG. 21 is a side elevation view of the bone anchor of FIG. 20;

FIG. 22 is a section view of the bone anchor of FIG. 21;

FIG. 23 is a perspective view of a first embodiment of the top cap for a polyaxial bone anchor;

FIG. 24 is a top view of the top cap of FIG. 23;

FIG. 25 is a side elevation view taken in a first radial direction of the top cap of FIG. 23;

FIG. 25A is a section view of the cam housing;

FIG. 25B is a section view of the bone anchor fitted within the cam housing;

FIG. 26 is a side elevation view taken in a second radial direction of the top cap of FIG. 23;

FIG. 27 is a side elevation view of the apparatus of FIG. 19 showing the bone anchor angulated within the apparatus and the bottom ring in an unlocked position;

FIG. 28 is a side elevation view of the apparatus of FIG. 19 showing the bone anchor angulated within the apparatus and the bottom ring in a locked position;

FIG. 29 is a perspective view of an embodiment of a polyaxial apparatus showing the bone anchor angulated and the bottom ring in the unlocked position and the upper wave cap in the unlocked position; and

FIG. 30 is a perspective view of an embodiment of a polyaxial apparatus showing the bone anchor angulated and the bottom ring in the locked position and the upper wave cap in the locked position.

FIG. 31 is a perspective view of an alternative embodiment showing a cam assembly external to the flanges shown in the locked position;

FIG. 32 is a side elevation view of the embodiment of FIG. 31;

FIG. 33 is a perspective view of the upper cap of the embodiment shown in FIG. 31;

FIG. 34 is a side elevation view of the upper cap shown in FIG. 33;

FIG. 35 is a perspective view of the embodiment shown in FIG. 31 shown in an unlocked position;

FIG. 36 is a side elevation view of the embodiment shown in FIG. 35;

FIG. 37 is a perspective view of a second alternative embodiment showing a cam assembly external to the flanges shown in the locked position;

FIG. 39 is a side elevation view of the embodiment of FIG. 37;

FIG. 39 is a perspective view of the upper cap of the embodiment shown in FIG. 37;

FIG. 40 is a side elevation view of the upper cap shown in FIG. 39;

FIG. 41 is a perspective view of the embodiment shown in FIG. 37 shown in an unlocked position;

FIG. 42 is a side elevation view of the embodiment shown in FIG. 41;

FIG. 43 is a perspective view of another embodiment of the invention showing a non-polyaxial anchor utilizing a threaded locking assembly to secure a rod;

FIG. 44 is a side view in a first radial direction of the embodiment shown in FIG. 43;

FIG. 45 is a perspective view of the apparatus of the embodiment shown in FIG. 43;

FIG. 46 is a side view in a first radial direction of the apparatus shown in FIG. 45;

FIG. 47 is a side view in a second radial direction of the apparatus shown in FIG. 45;

FIG. 48 is a top view of the apparatus shown in FIG. 45;

FIG. 49 is a perspective view of a polyaxial bone anchor for use with another embodiment of the invention;

FIG. 50 is a side view of the polyaxial bone anchor shown in FIG. 49;

FIG. 51 is a section view of the polyaxial bone anchor shown in FIG. 49;

FIG. 52 is a top view of the polyaxial bone anchor shown in FIG. 49;

FIG. 53 is a perspective view of a housing for use with the polyaxial bone anchor shown in FIG. 49;

FIG. 54 is a side view of the housing shown in FIG. 53;

FIG. 55 is a section view of the housing shown in FIG. 53;

FIG. 56 is a top view of the housing shown in FIG. 53;

FIG. 57 is a perspective view of a threaded locking assembly for use with several threaded embodiments of the invention;

FIG. 58 is a side view of the threaded locking assembly shown in FIG. 57; and

FIG. 59 is a top view of the threaded locking assembly shown in FIG. 57.

### DETAILED DESCRIPTION OF THE PREFERRED EMBODIMENT

While the present invention will be described more fully hereinafter with reference to the accompanying drawings, in which particular embodiments and methods are shown, it is to be understood from the outset that persons of ordinary skill in the art may modify the invention herein described while achieving the functions and results of this invention. Accordingly, the description which follows is to be understood as illustrative and exemplary of specific embodiments within the broad scope of the present invention and not as limiting the scope of the invention. In the following descriptions, like numbers refer to similar features or like elements throughout.

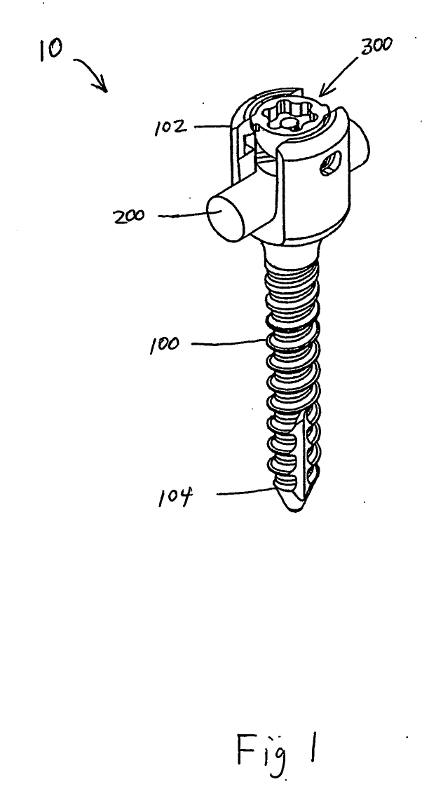

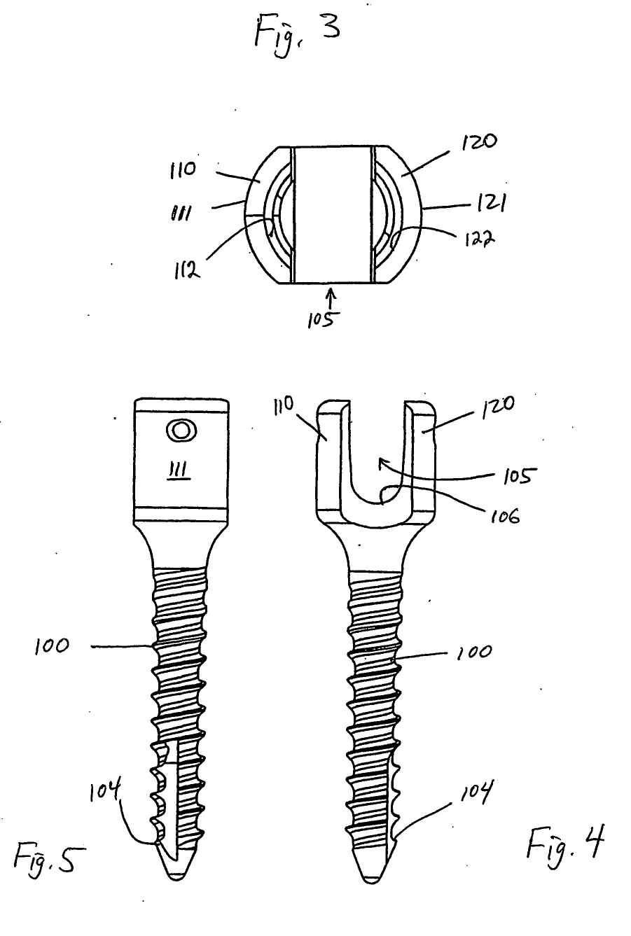

Ideally, an anchor apparatus for a spinal rod system will provide the surgeon with a device that rapidly and securely couples the spinal rod to the bone anchor. FIGS. 1-8 show an apparatus 10 according to a first non-polyaxial embodiment of the present invention. In general, the apparatus 10 generally comprises a bone anchor 100, a surgical rod 200, and a locking assembly 300. The bone anchor 100 can be any form of anchor suitable for the surgical technique employed, including, for example, a hook or a screw. In the figures, the bone anchor 100 is depicted as a screw for the sake of consistency. The bone anchor 100 comprises a proximal end 102 and a distal end 104. The distal end 104 engages a bone at a bone interface. The proximal end 102 engages the surgical rod 200 in a manner that will be described below.

Because the invention is directed to an anchor system, the following directional references will be employed herein. When discussing the bone anchor 100 or any component thereof, the word “longitudinal” shall refer to the direction of the longitudinal axis of the bone anchor 100. For embodiments where the bone anchor 100 is a screw, the longitudinal direction will therefore be in the direction of the length of the screw. In these cases, “proximal” or “proximally” shall mean in the direction of or toward the proximal end 102; and “distal” or “distally” shall mean in the direction of or toward the distal end 104. Similarly, the term “radially” shall refer to a direction that is perpendicular to the longitudinal axis of the bone anchor 100. When referring to the spine, however, the term “longitudinal” shall refer to a direction along the length of the spine.

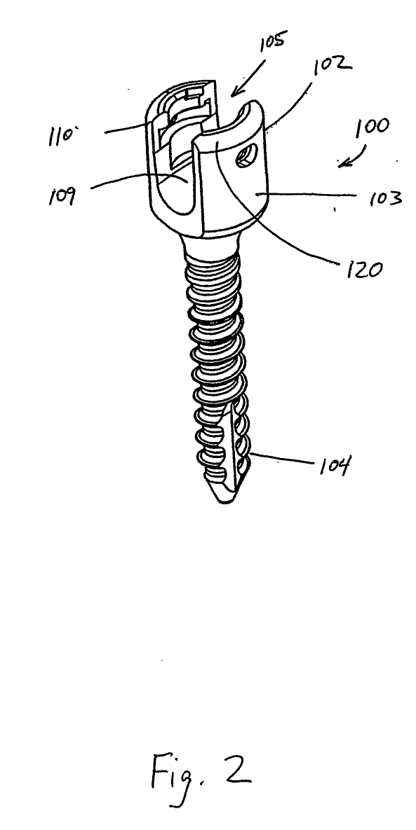

Referring to FIGS. 2-5, the proximal end 102 of the bone anchor 100 comprises preferably a partially cylindrical body 103 having a first flange 110 and a second flange 120 opposed to and spaced-apart from said first flange 110. The first flange 110 and the second flange 120 extend longitudinally in the proximal direction, and are separated by a channel 105 therebetween. A head 109 for engaging a driving instrument is preferably disposed within the partially cylindrical body 103.

First flange 110 has a convex outer surface 111 and a concave inner surface 112. Similarly, second flange 120 has a convex outer surface 121 and a concave inner surface 122. As referenced above, preferably the outer surfaces 111, 121 circumscribe a portion of a circle, with a gap coinciding with the channel 105, when viewed in the longitudinal direction, as best seen in FIGS. 3 and 4. In a similar fashion, preferably the inner surfaces 112, 122 circumscribe a portion of a smaller circle, with a gap coinciding with the channel 105. Other shapes, however, are possible, including polygonal shapes, ovoidal shapes, and so forth. The channel 105 is preferably a U-shaped channel disposed about the outer surfaces 111, 121, such that it creates a rod-receiving groove in a first radial direction. The channel 105 further comprises a bottom surface 106 that is dimensioned to receive a surgical rod. Preferably, the bottom surface 106 is semi-circular to receive the outer surface of a cylindrical rod 200.

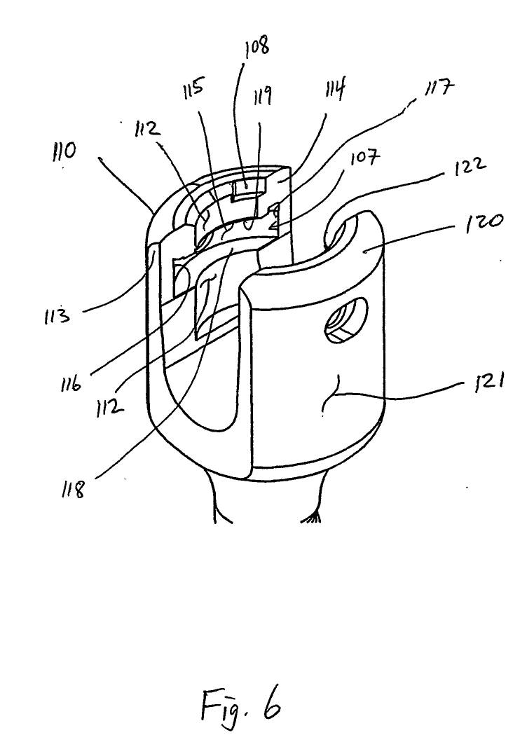

Referring now to FIGS. 6 and 7, first flange 110 further comprises an undercut 107 in the inner surface 112. The undercut 107 receives a portion of the locking assembly 300 (described below) and serves only to prevent the locking assembly 300 from exiting the head 109. Because most threads used in surgical procedures are standard, right-hand threads, the figures are drawn accordingly, and terminology such as “leading” and “trailing” are used accordingly. Obviously, if left-hand threads are used, the terminology can remain the same, but the reference numerals in the attached figures would need to be switched: leading structures would become trailing structures, and vice versa. For simplicity, standard threads are assumed.

First flange 110 further comprises a leading surface 113 and a trailing surface 114. Undercut 107 is preferably a partial cylindrical surface defining a groove. However, many other shapes are possible, including partially spherical, partially ovoidal, and so forth, it being more important that a radial groove be provided thereby. Undercut 107 further comprises an outer surface 115 having a leading edge 116 and a trailing edge 117. Outer surface 115 is preferably, though not necessarily, a partially cylindrical surface. In addition, undercut 107 further comprises a bottom surface 118 and a top surface 119.

Preferably, second flange 120 is identical to first flange 110, and so the following structures are comparable to those for the first flange 110, but are not shown in FIG. 6. As a result, second flange 120 further comprises a leading surface 123 and a trailing surface 124. Undercut 107 is essentially a partial cylindrical surface defining a groove. Undercut 107 further comprises an outer surface 125 having a leading edge 126 and a trailing edge 127. As state above, outer surface 125 is preferably, though not necessarily, a partially cylindrical surface. In addition, undercut 107 further comprises a bottom surface 128 and a top surface 129.

Because the undercut 107 extends radially through inner surfaces 112, 122 toward outer surfaces 111, 121, undercut 107 defines a diameter larger than the diameter of the inner surfaces 112, 122, but smaller than the diameter of the outer surfaces 111, 121.

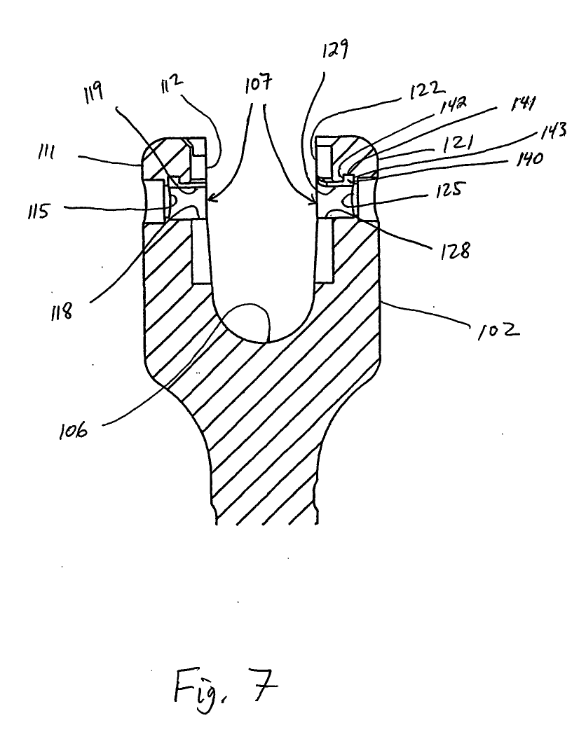

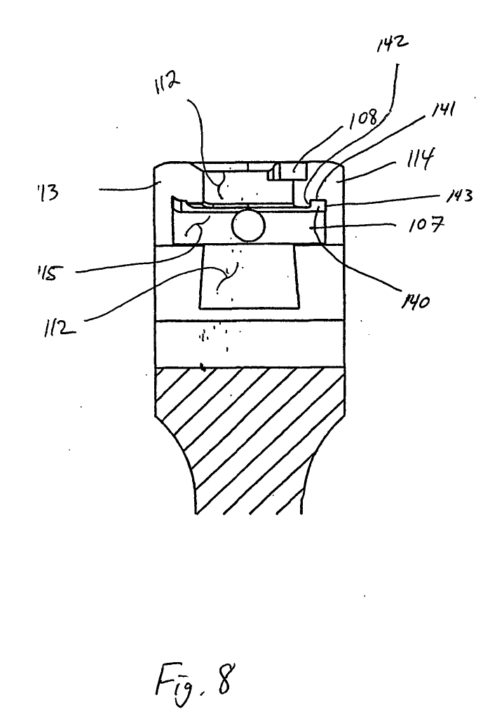

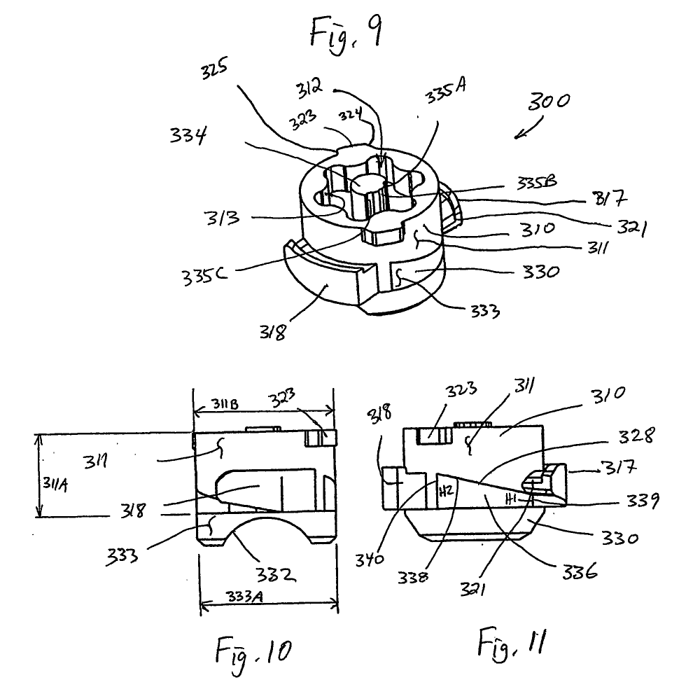

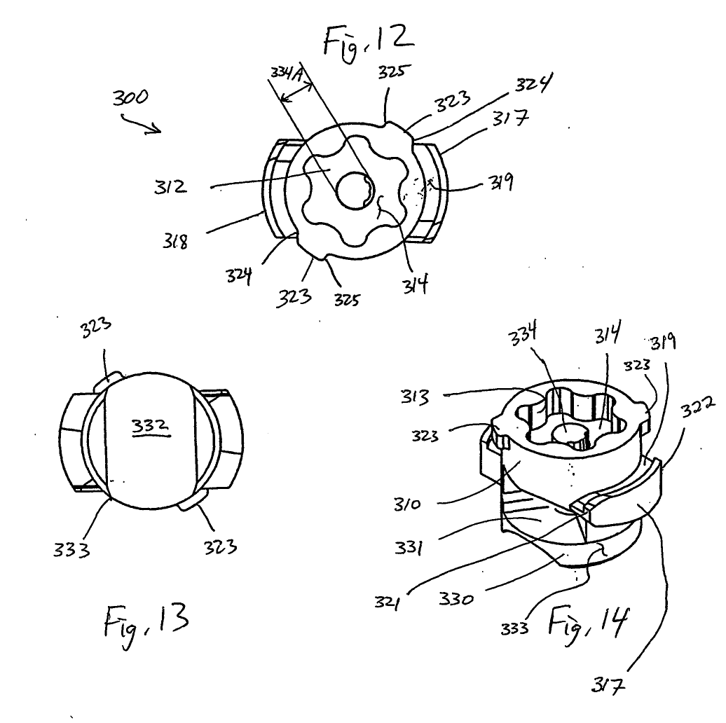

Referring now to FIGS. 9-18, a locking assembly 300 according to a first embodiment is depicted. The locking assembly 300 preferably comprises two parts: an upper cap 310 and a lower cap 330. The lower cap 330 according to this embodiment (FIG. 16) is a substantially cylindrical disk-like member having an upper surface 331 and a lower surface 332 and an outer surface 333 having a diameter 333A. A post 334 extends longitudinally away from the upper surface 331. The post 334 is substantially cylindrical and has a diameter 334A smaller than the diameter of the lower cap 330. Preferably three timing grooves 335A, 335B, and 335C (collectively, 335) are provided longitudinally along the post 334, as is shown in FIG. 9. Timing grooves 335 are preferably spaced 45 degrees apart.

Upper surface 331 further comprises opposed helical segments 336, 337. Helical segment 336 further comprises upper camming surface 338 having a leading edge 339 and a trailing edge 340. Upper camming surface 338 resides above the upper surface 331 a first height H1 at the leading edge and a second height H2 at the trailing edge, where H2 is greater than H1. Lower surface 332 is concave in a first radial direction so as to create a channel or saddle to engage an outer surface of a rod 200.

The upper cap 310 (FIG. 15) according to this embodiment is a generally cylindrical member having an outer surface 311 and a height 311A and a diameter 311B, and preferably having an inner cavity 312 therein for receiving a driving instrument (such as, a square driver, a pentagonal driver, a star driver, a hex driver, a Torx driver, a modified Torx driver, or any other geometric driving device that utilizes mating male/female members utilized to apply a torque). Inner cavity 312 therefore comprises sidewalls 313 (the number and shape of which are determined by the desired type of driving instrument) and a floor 314. The diameter 311B of the outer surface 311 is preferably the same as the diameter 333A of the outer surface 333 of lower cap 330.

Preferably, an opening 315 is disposed longitudinally in the floor 314 to receive the post 334. The opening 315 can take many shapes, depending on the shape of the desired post 334 and its resulting functions. However, most preferably the opening 315 is cylindrical with a projection 316 therein for mating with the timing grooves 335 in the post 334.

Referring again to the upper cap 310, it can be seen that the upper cap 310 further comprises flanges or wings protruding radially outwardly from the outer surface 311. In particular, first wing 317 and second wing 318 extend outwardly from the outer surface 311 in order to engage undercut 107, as will be described below. First wing 317 and second wing 318 are disposed about the outer surface 311 preferably 180 degrees apart. Additionally, upper cap 310 further comprises lower camming surfaces 328, preferably spaced ninety degrees out of phase with the first wing 317 and second wing 318, to engage the upper camming surfaces 338 of the lower cap 330 (as will be described below). Preferably, first wing 317 and second wing 318 are identical. Therefore, the following structural description will preferably apply to both first wing 317 and second wing 318.

First wing 317 comprises an upper surface 319 and a lower surface 320 having a leading edge 321 and a trailing edge 322. The upper surface 319 is inclined in the direction of the trailing edge 322.

In the preferred form of this embodiment (both polyaxial and non-polyaxial) depicted in FIGS. 9-18, the upper cap 310 further comprises structure to prevent splaying of the flanges 110, 120 or 410, 420. Because the locking assembly 300 uses camming surfaces 328, 338 to apply compression to the rod 200, forces can be created radially outwardly that tend to splay the flanges 110, 120 away from one another. Therefore, as best seen in FIG. 15, some modifications exist to prevent splaying.

First wing 317 and second wing 318 further comprise a ridge 326, 327 respectively located outwardly thereof and away from outer surfaces 311, 321, respectively. Ridge 326 comprises an upper surface 351, an outer surface 352, and an inner surface 353. Similarly (though not visible in the figures), ridge 327 comprises an upper surface 361, an outer surface 362, and an inner surface 363. Additionally, as best seen in FIGS. 7 and 8, undercut 107 further comprises a slot 140 adjacent the outer surfaces 115, 125, respectively. Slot 140 further comprises an upper surface 141, an inner surface 142 and an outer surface 143. Upon insertion of the upper cap 310 into the cylindrical body 103, 403, rotation of the upper cap 310 causes ridges 326, 327 to enter slots 140. Inner surface 353 of the ridge 326 engages inner surface 142 of slot 140 as the upper cap 310 is rotated, thus preventing splaying.

Additionally, upper cap 310 preferably comprises one or more stops 323. In the preferred embodiment, stops 323 comprise protuberances extending radially from the outer surface 311 of the upper cap 310, having a leading surface 324 and a trailing surface 325. A depression 108 is located near the trailing surface 114 of the first flange 110 or the trailing surface 124 of the second flange 120, or both. The depressions 108 are situated in such an orientation such that no more than 90 degrees of rotation is allowed once the upper cap 310 is inserted therein. In the preferred form of this embodiment, the stops 323 are inserted into the depression 108 at approximately fifty-five degrees from the first radial along the axis of the channel 105.

It should be noted that the upper cap 310 may have many arrangements and orientations of stops 323. For instance, a stop 323 may be alternatively fitted at the leading or trailing edge 321, 322 of the first wing 317 or second wing 318 such that, upon rotation of 90 degrees inside the undercut 107, the stop 323 contacts a mating surface within the undercut 107. Alternatively, the stop 323 may be provided by a combination of the timing grooves 335 of the lower cap 330 and the projection 316 of the upper cap 310, such that upon rotation of 90 degrees, the projection 316 contacts a terminal edge of one of the timing grooves 335. Additionally, depression 108 could be located substantially on one flange 110, 120 such that all or a majority of the rotation of 90 degrees occurs atop the flange. This would result in a fairly large depression 108. Many other variations are possible.

Upper cap 310 and lower cap 330 are preferably joined by inserting the post 334 into the opening 315, aligning the timing grooves 335 with the projection 316 in such a manner that the upper cap 310 is timed for three positions: 0, 45, and 90 degrees (first timing groove 335A, second timing groove 335B, and third timing groove 335C, respectively). Initial assembly of the upper cap 310 and lower cap 330, and indeed the manner in which the surgeon will receive the assembly, results in the projection 316 residing in first timing groove 335A. Preferably the top of the post 334 is then compressed, either mechanically or hydraulically, in order to “mushroom” the top. This prevents the post 334 from thereafter passing through the opening 315, which would separate the upper cap 310 from the lower cap 330.

The apparatus 10 is used typically with a surgical rod 200 having an outer surface 201 and a length 202. Any type of surgical rod is allowable for use. Typically, the rod will be a surgical grade stainless steel, titanium, titanium alloy, carbon fiber, memory metal, or other material suitable for implantation. The rod 200 is preferably cylindrical and may have texture or form on its outer diameter as well as a means for rotating the spine such as a hex on one or either end, flats along the rod, and so forth.

Now that the structure for a first non-polyaxial anchor embodiment has been described, attention is turned to the operation and use of the system according to this embodiment. The surgical site is prepared by the surgeon by first exposing the appropriate spinal area. The surgeon then drills or probes the bone for initial provision of anchor holes. Next, the surgeon obtains the bone anchor 100 and engages the driving instrument with the head 109. The bone anchor 100 is inserted into the prepared opening in the bone, and a torque is applied to the driving instrument, driving the bone anchor 100 into the bone until the bone anchor 100 is seated on the bone at the bone interface and the channel 105 is oriented in the desired direction. Numerous anchor assemblies can be inserted in this manner, and a rod 200, with proper contours, is then inserted into the respective channels 105.

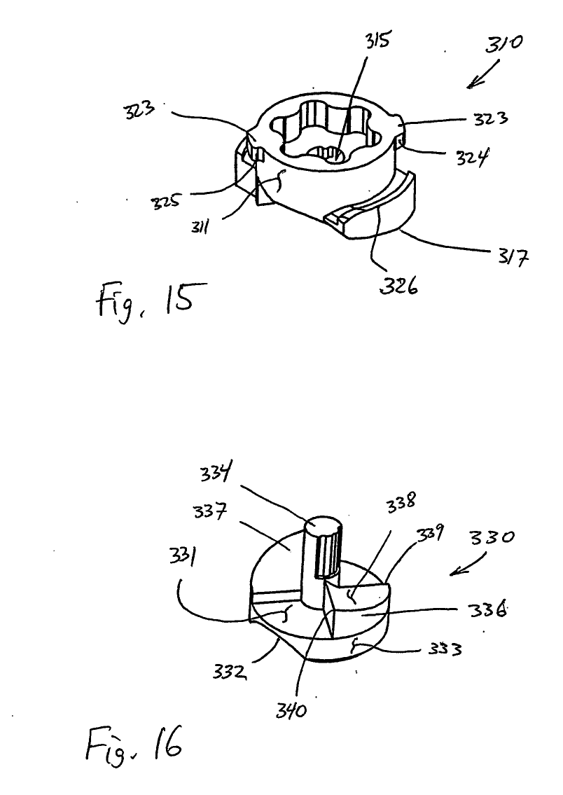

Next, the surgeon inserts a locking assembly 300 within each channel 105 atop the rod 200 such that the lower surface 332 of the lower cap 330 engages the outer surface 201 of the rod 200. This proper orientation results in the first wing 317 and second wing 318 aligned substantially in the same radial direction as the axis of the channel 105, and causes the stop 323 to reside in the depression 108, and causes projection 316 to reside in the first timing groove 335A (FIG. 17). The surgeon then engages a driving instrument with the inner cavity 312 of the upper cap 310 and applies a torque to begin rotating the upper cap 310 to quickly and positively lock the locking assembly 300 inside the cylindrical body 103 and the first flange 110 and second flange 120.

As torque is applied to the upper cap 310, this causes the upper camming surface 338 of the lower cap 330 and the lower camming surface 328 of the upper cap 310 to engage. This in turn causes leading edge 321 of the upper surface 319 and lower surface 320 of the first wing 317 and second wing 318 to enter the leading edges 116, 126 of the undercut 107, respectively. The application of additional torque causes lower camming surface 328 of the upper cap 310 to rotatingly traverse upon the upper camming surface 338 of the lower cap 330. Since the upper camming surface 338 and the lower camming surface 328 are inclined planes wrapped about a centroidal axis of rotation (thus, helixes), additional torque causes the upper cap 310 to continue to rotate about its axis and thus forces the lower cap 330 to translate longitudinally in the distal direction of the bone anchor 100. This translational movement compresses the lower surface 332 of the lower cap 330 onto the outer surface 201 of the rod 200. This compression is translated through the rod 200 and forces the outer surface 201 of the rod into compressive contact with the bottom surface 106 of the channel 105. Once the upper cap 310 has been turned 45 degrees, the projection 316 of the opening 315 of the upper cap 310 moves into the second timing groove 335B of the post 334. This provides the surgeon with tactile feedback that the upper cap 310 has been inserted halfway, and allows the surgeon to make any final minor adjustments of the rod/anchor interface.

Application of additional torque continues to rotate the upper cap 310 about the longitudinal axis and continues to cause lower camming surface 328 to traverse along upper camming surface 338, which in turn continues to translate the lower cap 330 longitudinally in the distal direction toward the bone. This continues to apply compressive force on the rod 200 as it seats within the channel 105 with increasing force. The surgeon continues to apply torque until the upper cap 310 has been turned 90 degrees. This then allows the projection 316 of the opening 315 of the upper cap 310 to seat into the third timing groove 335C of the post 334 (FIG. 18). At this point, the leading surface 324 of the stop 323 contacts the leading surfaces 113, 123 of the first flange 110 and second flange 120, respectively. This provides a positive mechanical stop and prevents any overtorque from being applied, thus preventing movement past 90 degrees. The seating of the projection 316 into the third timing groove 335C also provides a positive locking force tending to prevent rotation in the opposite, unlocking, direction.

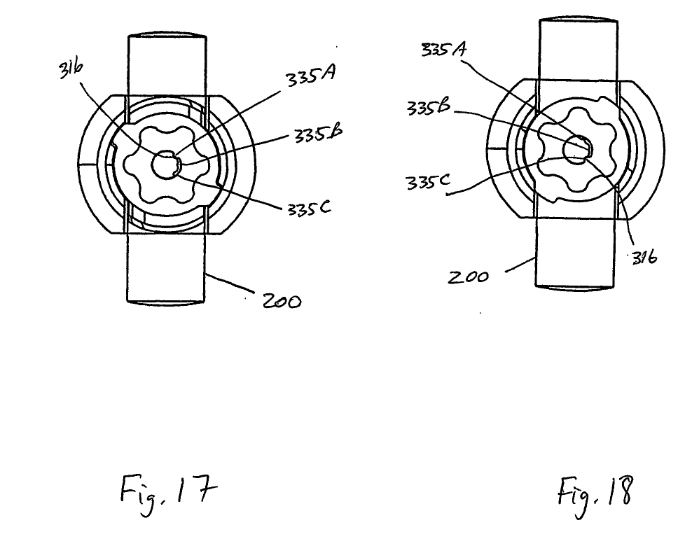

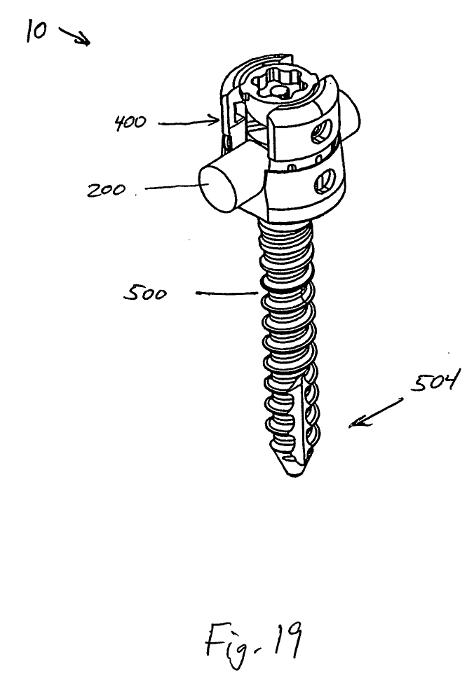

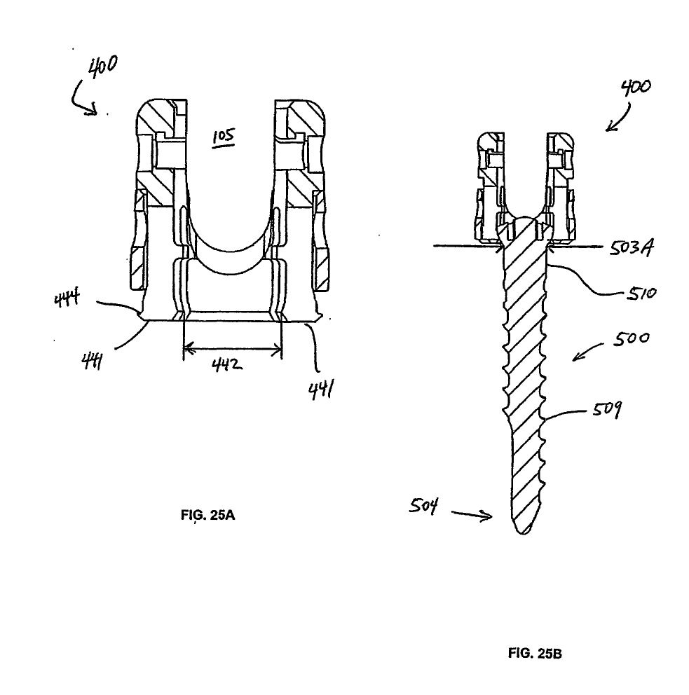

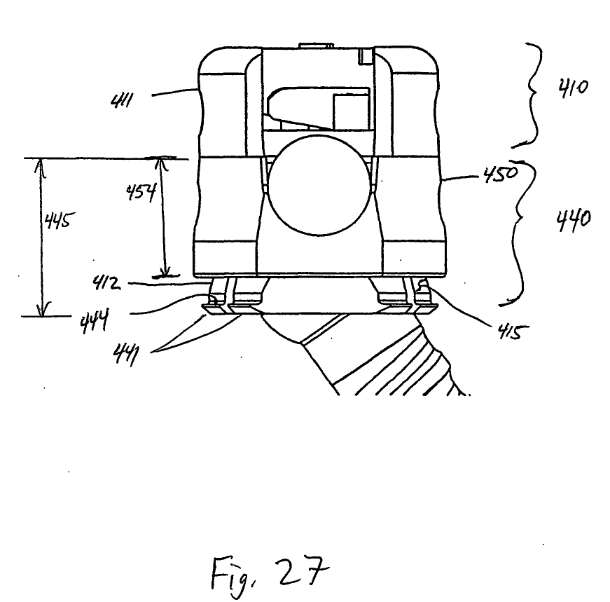

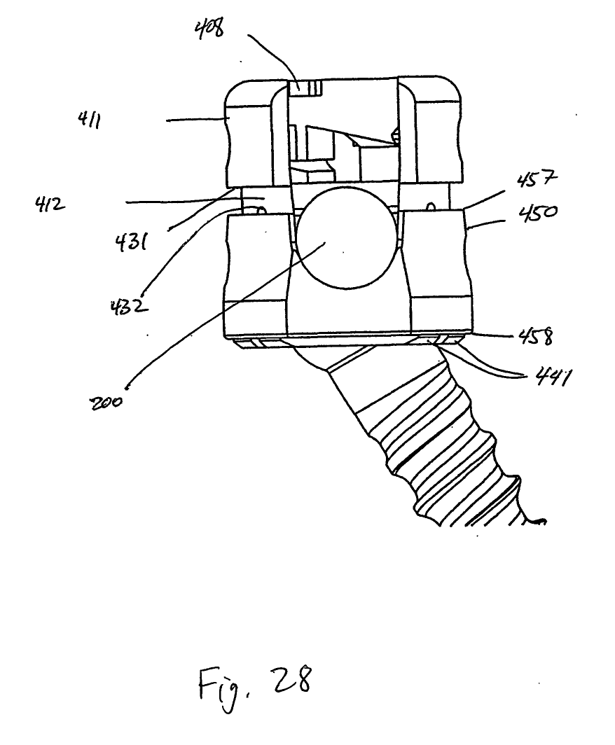

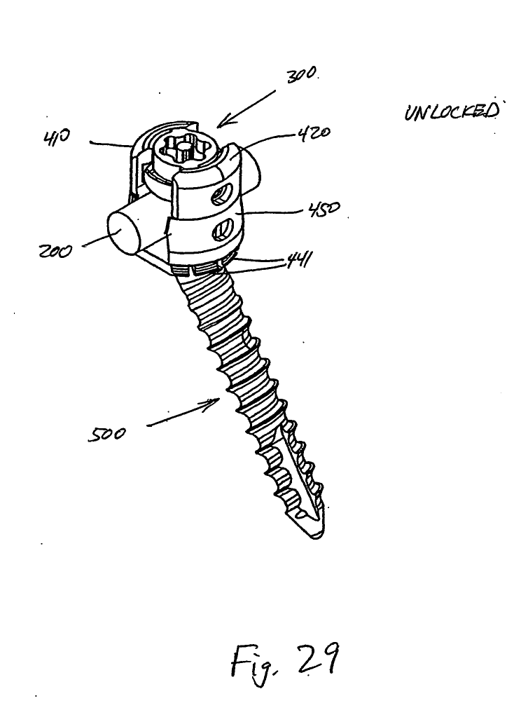

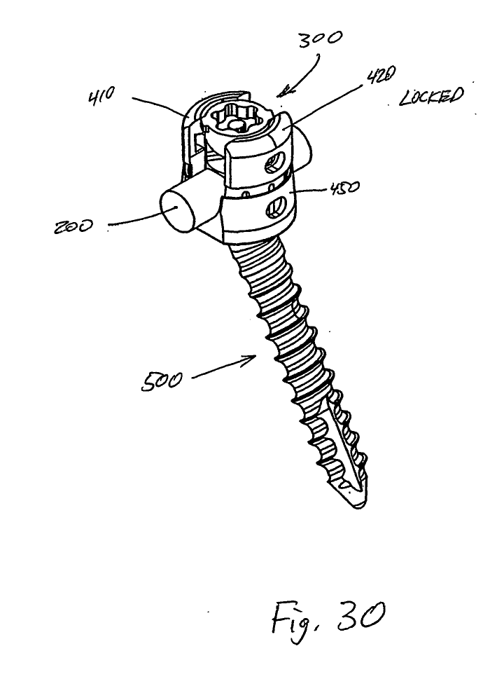

Now that the apparatus 10 has been described for use with a first non-polyaxial embodiment, the apparatus 10 will be described for use with polyaxial bone anchors 500 according to a second embodiment, depicted in FIGS. 19-30. Several modifications are present in this second polyaxial embodiment. As with the non-polyaxial bone anchor 100, the figures and descriptions depict a screw for the sake of simplicity.

Referring to FIGS. 19-22, the polyaxial bone anchor 500 omits the partially cylindrical body 103 of the nonpolyaxial embodiment. Instead, the polyaxial bone anchor 500 comprises a proximal end 502 and a distal end 504. At the proximal end is a head 501 having a lower surface 503 and an upper surface 505. At the distal end 504 are threads 509. A shank portion 510 is typically present between proximal end 502 and distal end 504. In the preferred polyaxial embodiment, the lower surface 503 is a spherical section. The spherical surface allows the anchor 500 to angulate at varying angles in a ball-and-socket manner, as will be described below. The lower surface 503 has a diameter 503A adjacent the shank 510 of the anchor 500 and a diameter 505A at the union of the upper surface 505 and the lower surface 503, wherein the diameter 505A is greater than the diameter 503A. Similarly, in the preferred polyaxial embodiment, the upper surface 505 is a conic section. The conic section is used to reduce the height of the head 501 compared to that of a known spherical or ball-shaped heads, while still allowing for the same amount of anchor/rod interface that would be possible with a ball-shaped head.

The upper surface 505 of the head 501 may also have an internal cavity 506 therein for engagement with a driving mechanism. Many forms of engagement geometries are possible. In the figures a modified Torx system is shown, having a center hub 507 having an upper surface 508 and a diameter 507A. The upper surface 508 maintains the conic section of the upper surface 505, and contacts the rod 200. The diameter of the center hub 507 is dependent upon the amount of angulation of the anchor 500 desired, as will be described below.

Referring to FIGS. 23-26, a housing 400 is provided to accept the rod 200 and join it with the polyaxial anchor 500. Housing 400 is akin to the partially cylindrical body 103 of the non-polyaxial anchor described above, with a few modifications. Indeed, housing 400 further comprises a partially cylindrical body 403 having a first flange 410 and a second flange 420 opposed to and spaced-apart from said first flange 410. The first flange 410 and the second flange 420 extend longitudinally in the proximal direction, and are separated by a channel 405 therebetween.

Referring to FIGS. 27-30, one modification present in the preferred form of this polyaxial embodiment resides in the structure of the outer surfaces of the flanges 410, 420. First flange 410 has an upper portion 411 and a lower portion 412, wherein an outer diameter 411A of the upper portion 411 first flange 410 and second flange 420 is greater than an outer diameter 412A of the lower portion 412, thus creating a step 431 at the junction of upper portion 411 and lower portion 412 (FIG. 28). Preferably upper portion 411 further comprises one or more slots 432 therein to receive and properly orient a bottom ring 450 (described below). Upper portion 411 has a convex outer surface 413 and a concave inner surface 414, in a manner similar to that of surfaces 111, 112 of first flange 110 in the preferred non-polyaxial embodiment.

Similarly, second flange 420 has an upper portion 421 and a lower portion 422 akin to the upper portion 411 and lower portion 412 of the first flange 410. Upper portion 421 has a convex outer surface 423 and a concave inner surface 424, in a manner similar to that of surfaces 121, 122 of second flange 120 in the preferred non-polyaxial embodiment. Upper portions 411, 421 also preferably have a depression 408 alcin to depression 108 in the non-polyaxial embodiment.

As referenced previously with respect to a non-polyaxial embodiment, preferably the outer surfaces 413, 423 circumscribe a portion of a circle, with a gap coinciding with the channel 405, when viewed in the longitudinal direction, as best seen in FIG. 25. In a similar fashion, preferably the inner surfaces 414, 424 circumscribe a portion of a smaller circle, with a gap coinciding with the channel 405, when viewed in an axial or longitudinal direction.

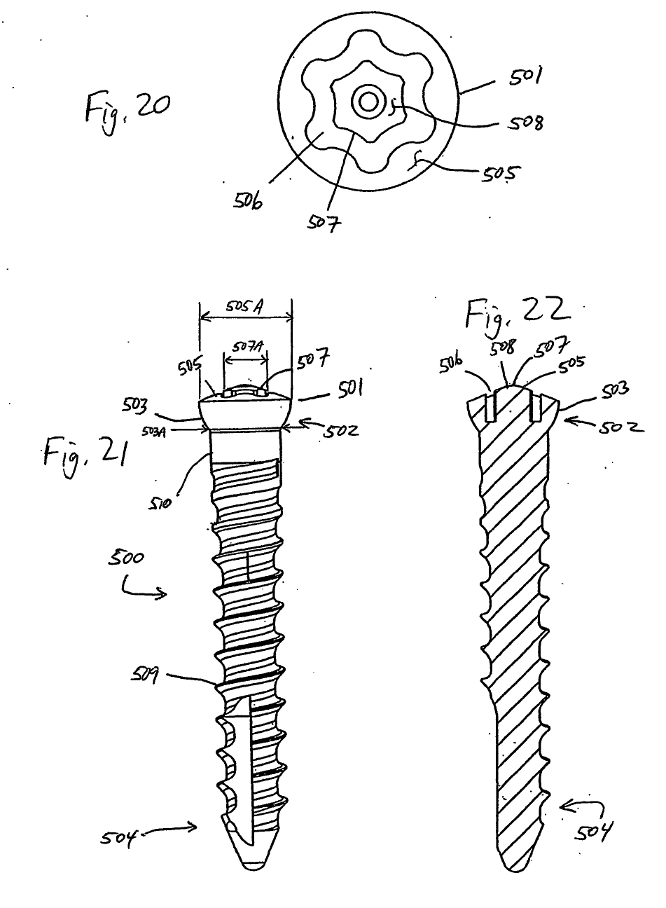

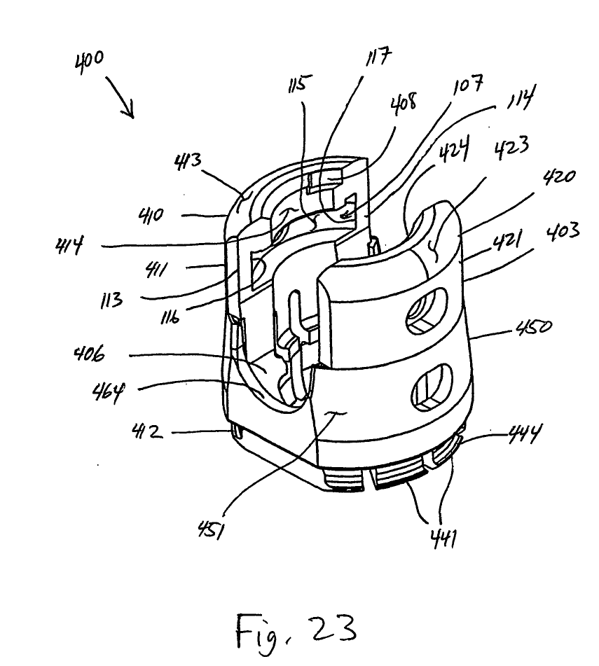

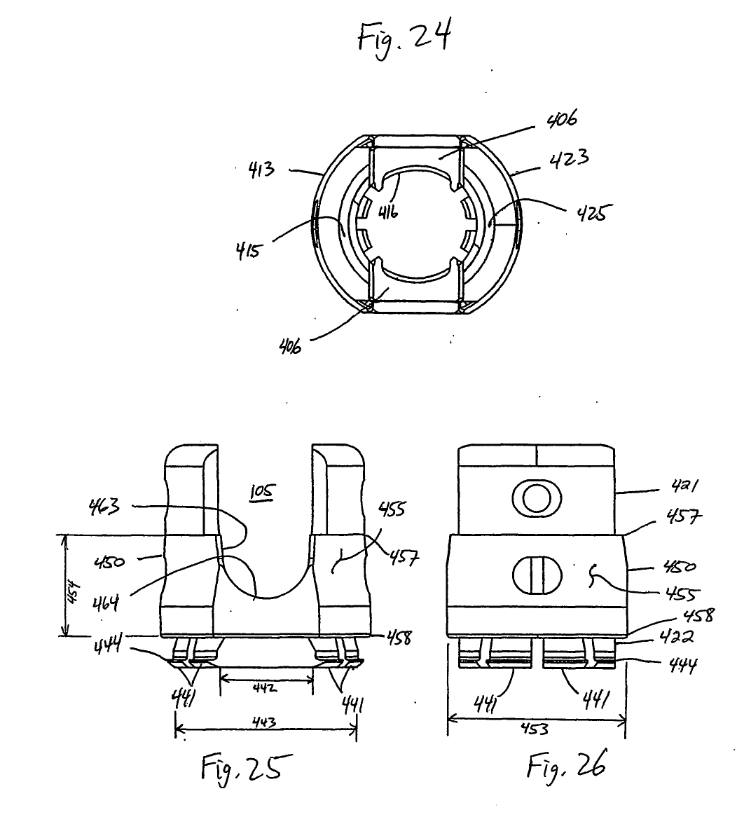

The lower portion 440 further comprises a generalized cylinder having a height 445 and having multiple resiliently opposed fingers 441 depending longitudinally in the distal direction. Lower portion 440 further comprises a convex outer surface 415 and a concave inner surface 416 (FIG. 24). Fingers 441 define an inner diameter 442 and an outer diameter 443. The inner diameter 442 is preferably the same as the diameter 503A of the lower surface 503 of the head 501 of the polyaxial bone anchor 500 (FIGS. 25, 25A, and 25B). Because there is a chamfer on the bottom of the head fingers, this allows the screw head 503 to be installed into the head as the fingers are forced outwards creating a large diameter due to the radial spring effect. This also means the outer ring must be up to allow this to occur. Fingers 441 also have a lip 444 on the outer surface thereof such that the diameter of the lip 444 is greater than the outer diameter 443. As an added feature of versatility, fingers 441 are dimensioned such that, upon application of a minimal amount of force in the longitudinal direction (which results in a spreading of the fingers 441), the lower portion 440 can be placed on the head 501 of the anchor 500 after the anchor 500 has been placed in the bone. In other words, two methods of insertion are possible. First, the anchor 500 can be inserted into the bone and then the housing 400 can be placed thereon. Second, the housing 400 can be placed on the anchor 500 and then the anchor/top cap assembly can be inserted into the bone.

The channel 405 is preferably a U-shaped channel disposed about the junction of outer surfaces 413, 415 and 423,425, such that it creates a rod-receiving groove in a first radial direction. The channel 405 further comprises a bottom surface 406 that is dimensioned to receive a surgical rod 200. Preferably, the bottom surface 406 is semi-circular to receive the outer surface of a cylindrical rod 200.

Referring again to FIGS. 27-30, when the anchor 500 is inserted into the partially cylindrical body 403 of the housing 400, the lower surface 503 seats on the inner diameter 442 of the fingers 441. Since the lower surface 503 is curvate, angulation of the anchor 100 is possible. This creates a sort of ball and socket arrangement. In the preferred embodiment, angulation of approximately 0 to 60 degrees included angle is possible.

Referring again to FIGS. 24-30, a further modification for the preferred form of this polyaxial embodiment is the addition of a bottom ring 450. Bottom ring 450 comprises a generalized open cylindrical body 451 having an inner diameter 452 (not visible in the figures), an outer diameter 453, a height 454, an outer surface 455, an inner surface 456, a proximal end 457, and a distal end 458. When assembled, the bottom ring 450 surrounds the lower portion 440 such that the inner surface 456 lies on the outside of the outer diameter 443 of the lower portion 440. Inner surface 456 is dimensioned such that bottom ring 450 can translate longitudinally about outer diameter 443 of the lower portion. Similarly, bottom ring 450 is dimensioned such that height 454 is less than height 445 of the lower portion 440.

Referring again to FIGS. 23 and 25, the outer surface 455 further comprises a U-shaped slot 463 having a bottom surface 464. In the locked position, the bottom surface 464 is adjacent the bottom surface 406 of the lower portion 440 so as to lockingly facilitate the rod 200.

The proximal end 457 preferably can have one or more tabs 459 (not shown) dimensioned and positioned for mating with slots 432 in the upper portion 430. Thus, when assembled, bottom ring 450 can translate longitudinally in a path defined by the combination of tabs 459 and slots 432. Two terminal positions are possible for the bottom ring 450, and bottom ring 450 can slide between these two positions. In an unlocked position (shown in FIGS. 17, 23, 25, 26, 27, and 29), the bottom ring 450 resides toward the proximal end of the lower portion 440, and tabs 459 reside within slots 432. In a locked position (shown in FIGS. 18, 19, 25B, 28, and 30), the bottom ring 450 resides at the distal end of the lower portion 440 such that the distal end 458 is in contact with lip 444. The distal end 458 terminates in a bottom surface 460 having an inner diameter 461 and an outer diameter 462. The inner diameter 461 is slightly less than the outer diameter 443 of the fingers 441. Thus, when the bottom ring 450 is translated longitudinally toward the bone, the inner diameter 461 applies a radial force directed inwardly on the fingers 441. As this occurs upon further translation of the bottom ring 450, the fingers 441 compress upon the lower surface 503 of the head 501 of the anchor.

The internal characteristics of the upper portion 430 are largely similar to those of the cylindrical body 403 of the non-polyaxial embodiment described above. For this reason, many reference numerals are duplicated for this embodiment. Thus, as shown in FIGS. 23-28, first flange 410 further comprises an undercut 107 in the inner surface 414. The undercut 107 receives a camming surface of the locking assembly 300 (described above). First flange 410 further comprises a leading surface 113 and a trailing surface 114. Undercut 107 is preferably a partial cylindrical surface defining a groove. Undercut 107 further comprises an outer surface 115 having a leading edge 116 and a trailing edge 117. Outer surface 115 is preferably, though not necessarily, a partially cylindrical surface. In addition, undercut 107 further comprises a bottom surface 118 and a top surface 119.

Preferably, second flange 420 is identical to first flange 410. As a result, second flange 420 further comprises a leading surface 123 and a trailing surface 124. Undercut 107 is essentially a partial cylindrical surface defining a groove. Undercut 107 further comprises an outer surface 125 having a leading edge 126 and a trailing edge 127. Outer surface 125 is preferably, though not necessarily, a partially cylindrical surface. In addition, undercut 107 further comprises a bottom surface 128 and atop surface 129.

Because the undercut 107 extends radially within inner surface 414,424, undercut 107 defines a partial cylindrical section having a diameter larger than the diameter of the inner surfaces 414, 424, but smaller than a diameter of the outer surfaces 413, 423.

The operation and use of the this embodiment of the polyaxial anchor assembly is very similar to that of the non-polyaxial embodiment described above, except that angulation of the anchor 500 is allowed with the polyaxial embodiment. The surgical site is prepared by the surgeon in the same manner. The housing 400 and bottom ring 450 are pre-connected prior to being provided to the surgeon, and the bottom ring 450 is in its unlocked position. Next, the surgeon obtains the bone anchor 500 and, preferably, inserts it into the partially cylindrical body 403 of the housing 400 until the lower surface 503 of the head 501 contacts the inner diameter 442 of the fingers 441. As noted above, the anchor 500 can be installed with or without the housing 400 pre-installed. The surgeon engages the driving instrument with the internal cavity 506 of the head 501. The bone anchor 500 is inserted into the prepared opening in the bone, and a torque is applied to the driving instrument, driving the bone anchor 500 into the bone until the bone anchor 500 is seated on the bone at the bone interface at the desired angulation. The housing 400 is then freely turned until the channel 405 is oriented in the desired direction. Numerous anchor assemblies can be inserted in this manner, and a rod 200, with proper contours, is then inserted into the respective channels 405. Once the top caps 400 have been properly positioned, the surgeon then pushes longitudinally on the bottom ring 450 until the inner diameter 461 forces the fingers 441 to move radially inward and the bottom surface 460 of the bottom ring 450 rests on lip 444 of the lower portion 440. This aligns bottom surface 464 with bottom surface 406 and locks the bone anchor 500 in the desired angulation.

Next, the surgeon inserts a locking assembly 300 within each channel 405 atop the rod 200 such that the lower surface 332 of the lower cap 330 engages the outer surface 201 of the rod 200. This proper orientation results in the first wing 317 and second wing 318 aligned substantially in the same first radial direction as the longitudinal axis of the channel 405, and causes the stop 323 to reside in the depression 408. The surgeon then engages a driving instrument with the inner cavity 312 of the upper cap 310 and applies a torque to rotate the upper cap 310 to easily and positively lock the locking assembly 300 inside the cylindrical body 403 and the first flange 410 and second flange 420.

As torque is applied to the upper cap 310, this causes the upper camming surface 338 of the lower cap 330 and the lower camming surface of the upper cap 310 to engage. This in turn causes leading edge 321 of the upper surface 319 and lower surface 320 of the first wing 317 and second wing 318 to enter the leading edges 116, 126 of the undercut 107, respectively. The application of additional torque causes lower camming surface 328 of the upper cap 310 to rotatingly traverse upon the upper camming surface 338 of the lower cap 330. Since the upper camming surface 338 and the lower camming surface 328 are inclined planes wrapped about a centroidal axis of rotation (helixes), additional torque causes the upper cap 310 to continue to rotate about its axis and thus force the lower cap 330 to translate longitudinally in the distal direction of the longitudinal axis of the bone anchor 500. This translational movement compresses the lower surface 332 of the lower cap 330 onto the outer surface 201 of the rod 200. This compression is translated through the rod 200 and forces the outer surface 201 of the rod into compressive contact with the bottom surface 464 of the channel 405 and in contact with the upper surface 505 of the head 501. Once the upper cap 310 has been turned 45 degrees, the projection 316 of the opening 315 of the upper cap 310 seats into the second timing groove 335B of the post 334. This provides the surgeon with tactile feedback that the upper cap 310 has been inserted halfway, and allows the surgeon to make any final minor adjustments of the rod/anchor interface.

Application of additional torque continues to rotate the upper cap 310 about the longitudinal axis and continues to cause lower camming surface 328 to traverse along upper camming surface 338, which in turn continues to translate the lower cap 330 longitudinally in the distal direction toward the bone. This continues to apply compressive force on the rod 200 as it seats within the channel 405 with increasing force. The surgeon continues to apply torque until the upper cap 310 has been turned 90 degrees. This then allows the projection 316 of the opening 315 of the upper cap 310 to seat into the third timing groove 335C of the post 334. At this point, the leading surface 324 of the stop 323 contacts the leading surfaces 113, 123 of the first flange 410 and second flange 420, respectively. This provides a positive mechanical stop and prevents any overtorque from being applied, thus preventing movement past 90 degrees. The seating of the projection 316 into the third timing groove 335C provides a positive locking force tending to prevent rotation in the opposite, unlocking, direction.

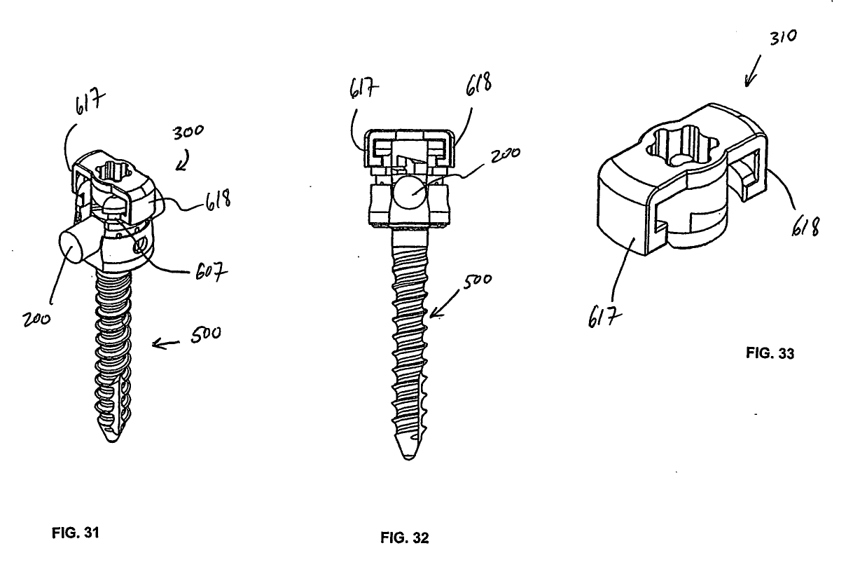

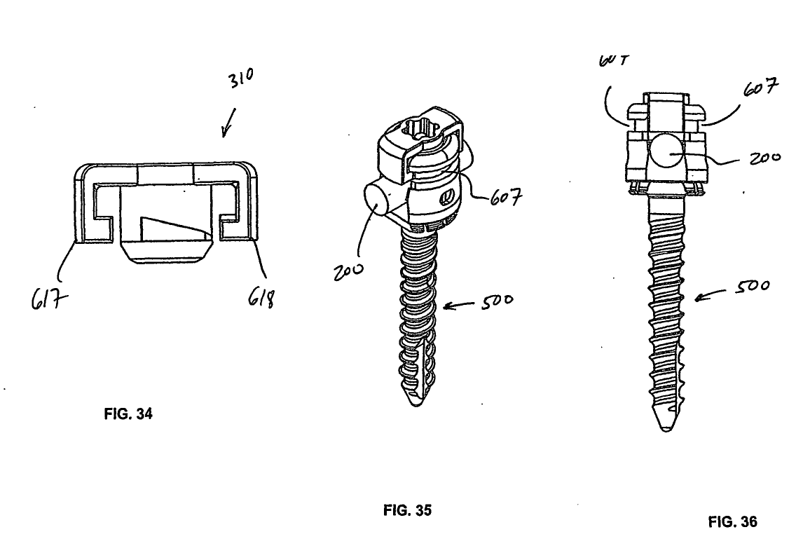

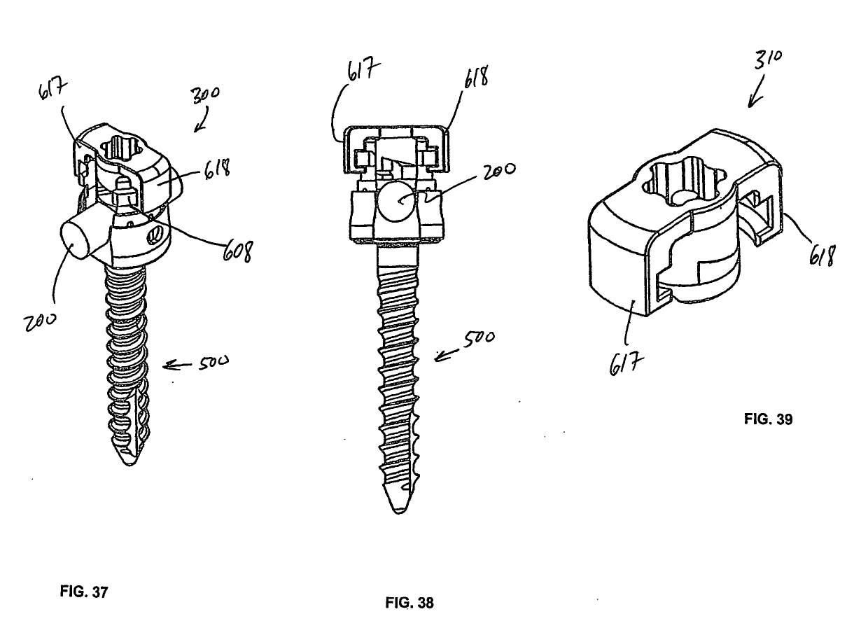

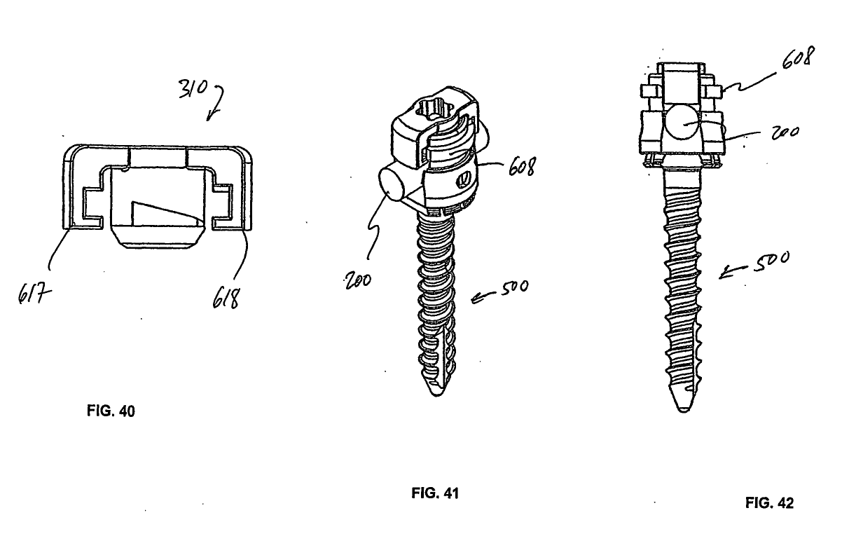

Referring now to FIGS. 31-42, another embodiment of the invention is shown. The locking assembly 300 can be external to the first flange 110 and second flange 120. In this embodiment, the flanges 110, 120 preferably contain a circumferential groove 607 in the convex outer surfaces 111, 121 thereof. A first wing 617 and a second wing 618 of the upper cap 310 enter the circumferential groove 607 upon activation of the locking assembly 300. Alternatively, the outer surfaces 111, 121 can contain a protrusion 608 for engaging a circumferential groove in the portion of the upper cap 310 that resides external to the outer surfaces 111, 121. In both cases, due to the external cam configuration, splaying of the flanges 110, 120 is easily remedied. In the external cam configurations, the camming action can be provided by an upper wave cap and lower cap as described above in reference to the internal configurations. However, the camming action can also be provided by the interaction of the first wing 617 and the second wing 618 and the circumferential groove 607. For instance, the circumferential groove itself can comprise an arcuate engagement slot for engaging opposed arcuate engagement surfaces (for example, a helical surface) on the first wing 617 and second wing 618. These and other methods of camming the wings to the head 501 are contemplated.

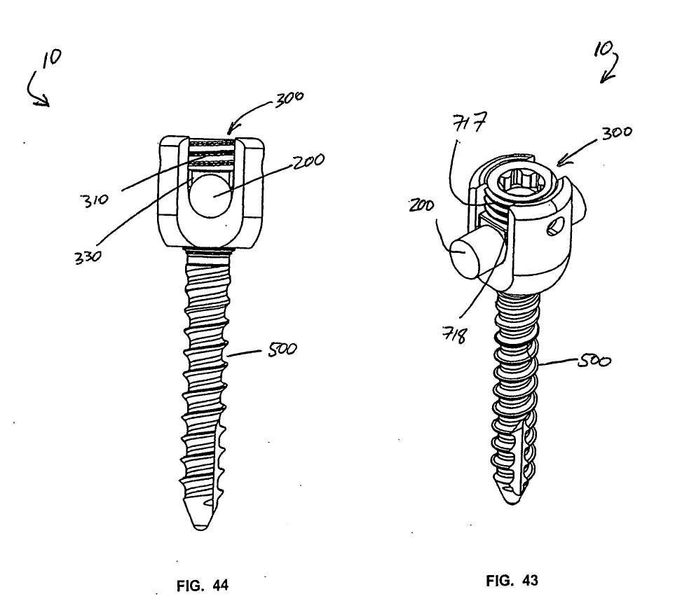

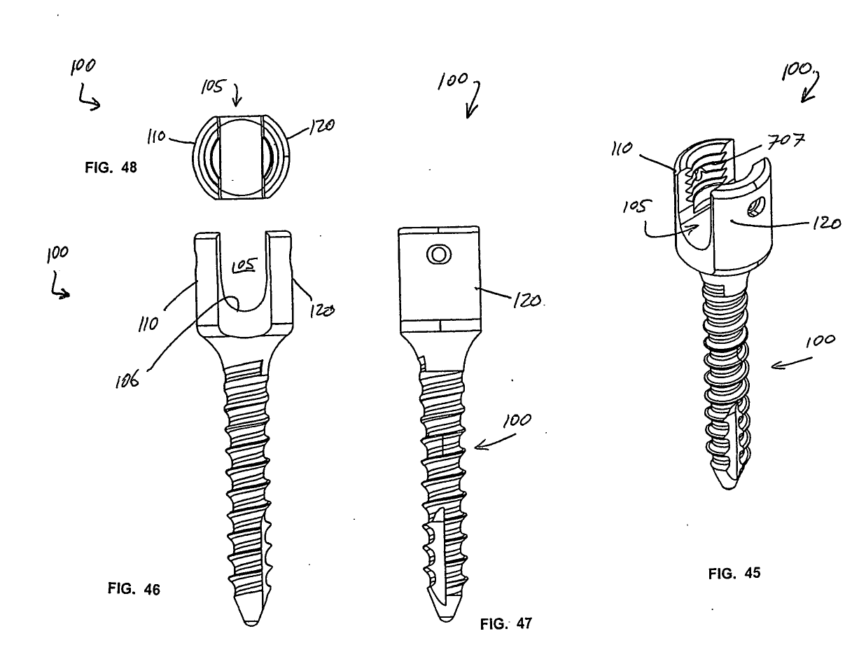

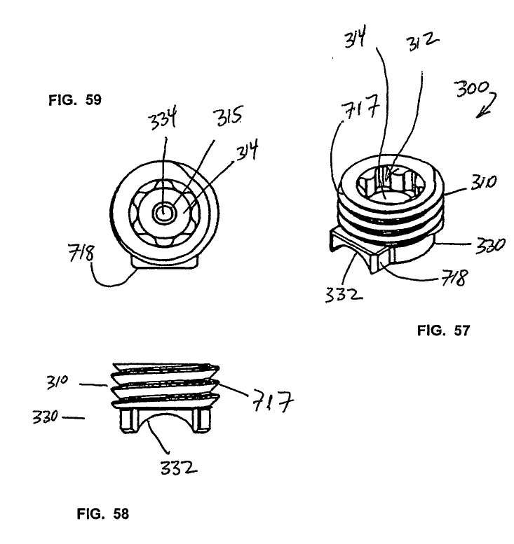

Referring now to FIGS. 43-59 another embodiment of both the polyaxial apparatus 10 and the non-polyaxial apparatus 10 is shown having a locking assembly 300 that utilizes a threaded upper cap 310. FIGS. 45-48 depict a non-polyaxial apparatus 10. FIGS. 43, 44, and 49-59 depict a polyaxial embodiment.

Referring now to FIGS. 45-48, the apparatus 10 is seen to be quite similar to that described above with regard to FIGS. 2-5. However, this embodiment utilizes a threaded locking assembly 300. The first flange 110 and second flange 120 have similar structure as that described above, but instead of comprising an undercut 107 in inner surface 112, the flanges 110, 120 comprise female threads 707. These threads 707 are for engagement by corresponding threads 717 on upper cap 310.

As stated, the locking assembly 300 of this embodiment comprises an upper cap 310 that is threaded, and a lower cap 330 that is similar to the lower cap 330 described previously. The upper cap 310 in this embodiment comprises threads 707 on its outer surface. Preferably the threads 707 are of the buttress type, but many forms of thread are possible. The upper cap 310 further comprises an inner cavity 312 therein that can have sidewalls 313 and floor 314 as described previously. The floor 314 can likewise contain an opening 315 to receive post 334. Projection 316 and timing grooves 335 in post 334 are optional.

The lower cap 330 comprises upper surface 331 and lower surface 332 wherein upper surface 331 is generally flat and wherein lower surface 332 is generally cylindrical (e.g., forming a saddle) to engage an outer surface of a rod 200. In the preferred form of this embodiment, the lower cap 330 preferably comprises an extension 718 of the lower surface 332. This extension 718 is dimensioned to fit within the channel 105 and to properly orient the locking assembly 300 during implantation. Ideally, the threads 717 can include single or double starts that can be timed with the lower surface 332 of the lower cap 330, and also with the extension 718. In this manner, once a rod is placed into the channel 105, the locking assembly 300 is inserted into the channel 105 atop the rod 200. Because the threads 717 are timed in conjunction with the extension 718 and threads 707 of the anchor 100, this prevents cross threading that is common in many devices.

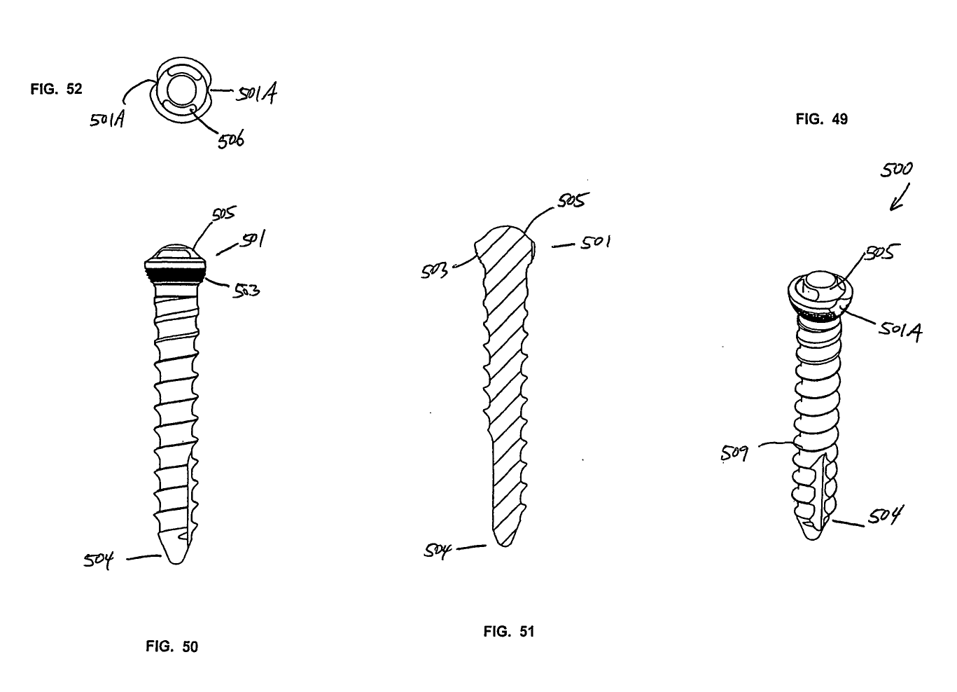

Referring now to FIGS. 43, 44, and 49-59, another embodiment of a polyaxial anchor 10 is shown, this embodiment likewise includes a threaded locking assembly 300 akin to that just described. With particular reference to FIGS. 49-52, in this polyaxial embodiment slight modifications are included in the anchor 500. In particular, the head 501 comprises one or more depression 501A about its circumference. The upper surface 505 optionally is conically shaped and is convex on its top and tapers to a slight concavity at its outer circumference.

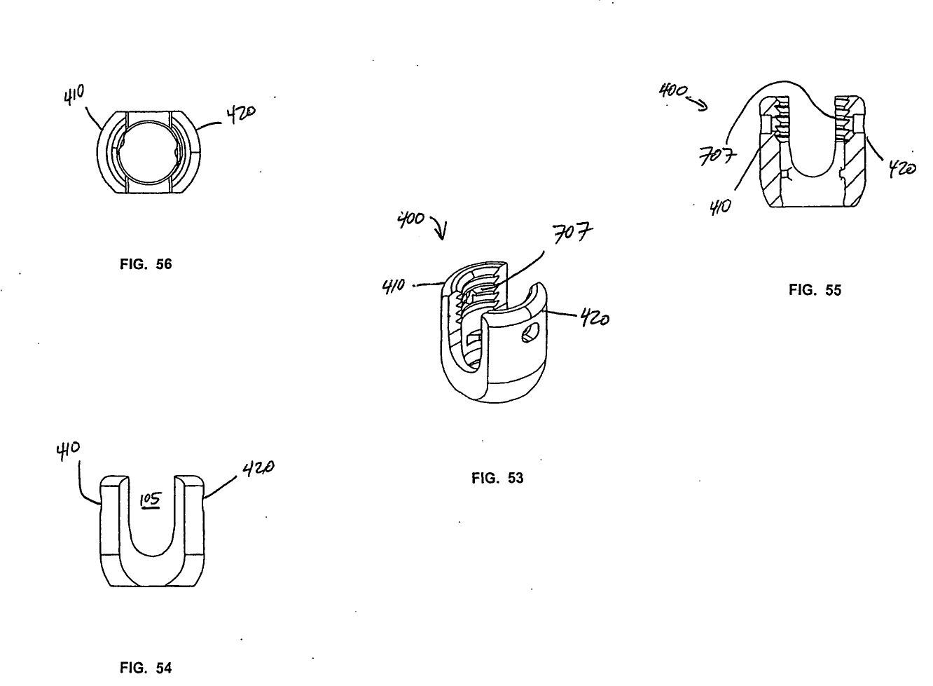

Referring now to FIGS. 53-56, a housing 400 of this embodiment is shown. The housing 400 is similar in many respects to the housing 400 previously described, with several notable exceptions. The first flange 410 and second flange 420 do not contain the undercut 107. Rather, as explained, the flanges 410, 420 comprise female threads 707 to receive the threads 717 of the upper cap 310. Due to the presence of the threads 707, this embodiment does not comprise the structural details related to the lower portion 440, fingers 441, and so forth. This embodiment instead comprises a single part housing 400 for receiving the anchor 500. The housing 400 does include a convex outer surface 413 and a concave inner surface 414, in similar fashion to previously described embodiments. In this embodiment, however, the inner surface 413 further comprises one or more appurtenance 713 protruding radially inwardly. This appurtenance 713 serves to engage the depression 501A in the head 501 of the anchor 500, thus forcing the anchor 500 to be inserted therein in a particular orientation. Once the depression 501A passes by the appurtenance 713, the anchor 500 is free to rotate. Upon any rotation thereafter, the depression 501A will not line up with the appurtenance 713, and therefore the anchor will not readily be removed from the housing 400.

While there has been described and illustrated particular embodiments of a novel spinal rod anchor assembly, and in particular, one that provides a quick-connect release, it will be apparent to those skilled in the art that variations and modifications may be possible without deviating from the broad spirit and principle of the present invention, which shall be limited solely by the scope of the claims appended hereto.

What is claimed is:

1. A bone fixation apparatus for a rod system comprising:
a bone anchor having a proximal end for engaging a driving device and a distal end for engaging a bone;
a housing coupled to said proximal end of said bone anchor having opposed spaced-apart flanges extending longitudinally defining a channel therebetween for receiving a rod, said flanges having an outer surface and an inner surface having female threads therein;
a locking assembly for locking said surgical rod to said housing in contact with said proximal end of said bone anchor;
wherein said locking assembly further comprises an upper cap and a lower cap joined by a post and rotatable relative one to the other, wherein said upper cap is a generally cylindrical member having an outer surface containing threads thereon and an inner cavity and an upper surface and a lower surface and wherein said inner cavity further comprises an opening to receive said post; and wherein said lower cap comprises an upper surface and a lower semi-cylindrical surface for engaging said rod; and
wherein said rod is insertable into said channel of said housing and said locking assembly is thereafter insertable into said channel of said housing, and wherein a driving instrument is insertable into said inner cavity of said upper cap and rotates said upper cap relative to said lower cap about said post, thereby engaging said threads, thus causing said locking assembly to translate longitudinally toward said distal end of said anchor into a forcing relationship with said rod, thus forcing said rod into a locking relationship with said proximal end of said anchor.

2. The bone fixation system of claim 1 wherein said bone anchor is a screw.

3. The bone fixation apparatus of claim 2 wherein said housing is fixed to said proximal end of said bone anchor so as to prevent angulation of said bone anchor.

4. The bone fixation system of claim 3 wherein said lower cap further comprises an extension of said lower semi-cylindrical surface dimensioned to reside within said channel.

5. The bone fixation system of claim 4 wherein said threads on said upper cap and said threads in said flanges further comprise at least one start thread timed to properly engage each other when said extension is located within said channel.

6. The bone fixation system of claim 1 wherein said bone anchor is a hook.

7. The bone fixation apparatus of claim 6 wherein said housing is fixed to said proximal end of said hook so as to prevent angulation thereof.

8. The bone fixation system of claim 7 wherein said lower cap further comprises an extension of said lower semi-cylindrical surface dimensioned to reside within said channel.

9. The bone fixation system of claim 8 wherein said threads on said upper cap and said threads in said flanges further comprise at least one start thread timed to properly engage each other when said extension is located within said channel.

10. The bone fixation system of claim 1 wherein said bone anchor is a polyaxial screw further comprising a head disposed at said distal end having a conic or aspheric upper surface and a partially spherical lower surface and defining a periphery at a junction of said upper and lower surfaces.

11. The bone fixation system of claim 10 wherein said lower cap further comprises an extension of said lower semi-cylindrical surface dimensioned to reside within said channel.

12. The bone fixation system of claim 11 wherein said threads on said upper cap and said threads in said flanges further comprise at least one start thread timed to properly engage each other when said extension is located within said channel.

13. The bone fixation system of claim 12 wherein said proximal end of said polyaxial screw further comprises a depression disposed about said periphery.

14. The bone fixation system of claim 13 wherein said inner surface of said flanges further comprise an appurtenance projecting substantially radially inwardly.

15. The bone fixation system of claim 14 wherein said polyaxial screw is removable from said housing when said depression aligns with said appurtenance, but is not removable when said depression is not aligned with said appurtenance.

16. A bone fixation apparatus for a rod system comprising:
a bone anchor having a proximal end for engagement with a driving device and a distal end for engagement with a bone;
a housing coupled to said proximal end of said bone anchor having opposed spaced-apart flanges extending longitudinally and having a channel therebetween to receive a surgical rod therein, each flange having an outer surface and an inner surface and an undercut within said inner surface;
a locking assembly for locking said surgical rod to said housing in contact with said proximal end of said bone anchor, further comprising an upper cap and a lower cap joined by a post and rotatable relative one to the other, wherein said upper cap is a generally cylindrical member having an outer surface and an inner cavity and an upper surface and a lower surface having camming surfaces thereon and wherein said inner cavity further comprises an opening to receive said post; wherein said outer surface has a first wing and a second wing projecting radially outwardly therefrom for insertion into said undercut to prevent removal of said locking assembly; and wherein said lower cap further comprises an upper surface having camming surfaces thereon for engagement with said camming surfaces of said upper cap and a lower semi-cylindrical surface for engaging said rod;
wherein said rod is insertable into said channel of said housing and said locking assembly is thereafter insertable into said channel of said housing, and wherein a driving instrument is insertable into said inner cavity of said upper cap and rotates said upper cap relative to said lower cap about said post, thereby moving said camming surfaces of said upper cap relative to said camming surfaces of said lower cap, thus causing said lower cap to translate longitudinally toward said distal end of said anchor into a forcing relationship with said rod, thus forcing said rod into a locking relationship with said proximal end of said anchor.

17. The bone fixation apparatus of claim 16 wherein said bone anchor is a screw.

18. The bone fixation apparatus of claim 17 wherein said housing is fixed to said proximal end of said bone anchor so as to prevent angulation of said bone anchor.

19. The bone fixation apparatus of claim 18 wherein said undercut further comprises a slot adjacent thereto disposed radially outwardly of said undercut having an inner surface and an outer surface and longitudinal dimension greater than that of said undercut.

20. The bone fixation apparatus of claim 19 wherein said first wing and said second wing further comprise ridges located outwardly thereof.

21. The bone fixation apparatus of claim 20 wherein said ridges engage said inner surfaces of said slots in said flanges to prevent splaying.

22. The bone fixation apparatus of claim 21 wherein said post further comprises a first timing groove and a second timing groove disposed longitudinally thereon.

23. The bone fixation apparatus of claim 22 wherein said upper cap further comprises a projection in said opening.

24. The bone fixation apparatus of claim 23 wherein said upper cap and said first and second timing grooves are timed such that said projection mates with said first timing groove when said locking assembly is in an unlocked position and mates with said second timing groove when said locking assembly is rotated to a locked position.

25. The bone fixation apparatus of claim 24 wherein said upper cap further comprises a stop disposed adjacent said upper surface thereof and projecting radially outwardly from said outer surface of said upper cap, said stop being located out of phase with said wings.

26. The bone fixation apparatus of claim 25 wherein said housing further comprises a depression disposed about said inner surface of said flanges.

27. The bone fixation apparatus of claim 26 wherein said stop engages said depression when said upper cap is rotated into a locked position.

28. The bone fixation apparatus of claim 16 wherein said bone anchor is a hook.

29. The bone fixation apparatus of claim 28 wherein said housing is fixed to said proximal end of said bone anchor so as to prevent angulation of said bone anchor.

30. The bone fixation apparatus of claim 29 wherein said undercut further comprises a slot adjacent thereto disposed radially outwardly of said undercut having an inner surface and an outer surface and longitudinal dimension greater than that of said undercut.

31. The bone fixation apparatus of claim 30 wherein said first wing and said second wing further comprise ridges located outwardly thereof.

32. The bone fixation apparatus of claim 31 wherein said ridges engage said inner surfaces of said slots in said flanges to prevent splaying.

33. The bone fixation apparatus of claim 32 wherein said post further comprises a first timing groove and a second timing groove disposed longitudinally thereon.

34. The bone fixation apparatus of claim 33 wherein said upper cap further comprises a projection in said opening.

35. The bone fixation apparatus of claim 34 wherein said upper cap and said first and second timing grooves are timed such that said projection mates with said first timing groove when said locking assembly is in an unlocked position and mates with said second timing groove when said locking assembly is rotated to a locked position.

36. The bone fixation apparatus of claim 35 wherein said upper cap further comprises a stop disposed adjacent said upper surface thereof and projecting radially outwardly from said outer surface of said upper cap, said stop being located out of phase with said wings.

37. The bone fixation apparatus of claim 36 wherein said housing further comprises a depression disposed about said inner surface of said flanges.

38. The bone fixation apparatus of claim 37 wherein said stop engages said depression when said upper cap is rotated into a locked position.

39. The bone fixation apparatus of claim 17 wherein said housing further comprises a separable body for lockable attachment to said proximal end of said screw.

40. The bone fixation apparatus of claim 39 wherein said separable body further comprises an upper portion having an outer diameter and an inner diameter and a lower portion having an outer diameter and an inner diameter and wherein said outer diameter of said lower portion is less than said outer diameter of said upper portion.

41. The bone fixation apparatus of claim 40 wherein said lower portion further comprises fingers oriented longitudinally and extending from said upper portion terminating at a distal end, wherein said fingers receive said proximal end of said screw in varying orientations.

42. The bone fixation apparatus of claim 41 wherein said distal end of said fingers define an inner diameter and an outer diameter.

43. The bone fixation apparatus of claim 42 wherein said lower portion further comprises a ring disposed externally on said fingers and slideable from an unlocked position to a locked position.

44. The bone fixation apparatus of claim 43 wherein said inner diameter of said ring at a bottom end thereof is slightly less than said outer diameter of said fingers such that as said ring is slid from said unlocked position to said locked position, said ring imparts a radial force on said fingers, thus locking said proximal end of said screw in a given orientation.

45. The bone fixation apparatus of claim 44 wherein said undercut further comprises a slot adjacent thereto disposed radially outwardly of said undercut having an inner surface and an outer surface and longitudinal dimension greater than that of said undercut.

46. The bone fixation apparatus of claim 45 wherein said first wing and said second wing further comprise ridges located outwardly thereof.

47. The bone fixation apparatus of claim 46 wherein said ridges engage said inner surfaces of said slots in said flanges to prevent splaying.

48. The bone fixation apparatus of claim 47 wherein said post further comprises a first timing groove and a second timing groove disposed longitudinally thereon.

49. The bone fixation apparatus of claim 48 wherein said upper cap further comprises a projection in said opening.

50. The bone fixation apparatus of claim 49 wherein said upper cap and said first and second timing grooves are timed such that said projection mates with said first timing groove when said locking assembly is in an unlocked position and mates with said second timing groove when said locking assembly is rotated to a locked position.

51. The bone fixation apparatus of claim 50 wherein said upper cap further comprises a stop disposed adjacent said upper surface thereof and projecting radially outwardly from said outer surface of said upper cap, said stop being located out of phase with said wings.

52. The bone fixation apparatus of claim 51 wherein said housing further comprises a depression disposed about said inner surface of said flanges.

53. The bone fixation apparatus of claim 52 wherein said stop engages said depression when said upper cap is rotated into a locked position.

54. A bone fixation apparatus for a rod system comprising:
a bone anchor having a proximal end for engagement with a driving device and a distal end for engagement with a bone;
a housing coupled to said proximal end of said bone anchor having opposed spaced-apart flanges extending longitudinally and having a channel therebetween to receive a surgical rod therein, each flange having an outer surface and an inner surface, said outer surface having a circumferential groove therein;
a locking assembly for locking said surgical rod to said housing in contact with said proximal end of said bone anchor, further comprising an upper cap and a lower cap joined by a post and rotatable relative one to the other, wherein said upper cap is a generally cylindrical member having an outer surface and an inner cavity and an upper surface and a lower surface having camming surfaces thereon and wherein said inner cavity further comprises an opening to receive said post; wherein said outer surface has a first wing and a second wing projecting radially outwardly therefrom for insertion into said circumferential groove in said outer surface of said housing to prevent removal of said locking assembly;
and wherein said lower cap further comprises an upper surface having camming surfaces thereon for engagement with said camming surfaces of said upper cap and a lower semi-cylindrical surface for engaging said rod;
wherein said rod is insertable into said channel of said housing and said locking assembly is thereafter insertable into said channel of said housing, and wherein a driving instrument is insertable into said inner cavity of said upper cap and rotates said upper cap relative to said lower cap about said post, thereby moving said camming surfaces of said upper cap relative to said camming surfaces of said lower cap, thus causing said lower cap to translate longitudinally toward said distal end of said anchor into a forcing relationship with said rod, thus forcing said rod into a locking relationship with said proximal end of said anchor.
